(19) United States
(12) Patent Application Publication
(10) Pub. No.: US 2021/0265063 A1
(43) Pub. Date: Aug. 26, 2021

(54) RECOMMENDATION SYSTEM FOR MEDICAL OPINION PROVIDER

(71) Applicant: International Business Machines Corporation, Armonk, NY (US)

(72) Inventors: Panagiotis Karampourniotis, Cambridge, MA (US); Daniel M. Gruen, Newton, MA (US); Amarendra Das, Cambridge, MA (US)

(21) Appl. No.: 16/802,053

(22) Filed: Feb. 26, 2020

(51) Int. Cl. G16H 50/70; G16H 70/20; G06Q 40/08; G16H 10/60; G06Q 30/02; G06N 5/04; G06N 20/00; G06F 16/26; G06F 40/30

(52) U.S. Cl. CPC G16H 50/70; G16H 70/20; G06Q 40/08; G16H 10/60; G06F 40/30; G06N 5/04; G06N 20/00; G06F 16/26; G06Q 30/0204

(57) ABSTRACT

Provided is a method, computer program product, and system for recommending a patient obtain a second medical opinion (SMO) from a second healthcare provider. A processor may collect a first set of attributes associated with a first healthcare provider, the first healthcare provider having identified a first medical opinion for treating a medical condition of a patient. The processor may identify a plurality of healthcare providers that have treated a similar medical condition for one or more other patients. The processor may compare the first set of attributes to a plurality of attributes related to the plurality of healthcare providers. The processor may identify a second healthcare provider based on analyzing similarities between a second set of attributes related to the second healthcare provider and the first set of attributes. Once identified, the processor may provide a recommendation to the patient to obtain a SMO from the second healthcare provider.

### BACKGROUND

The present disclosure relates generally to the field of computer systems, and more specifically, to a system for recommending a patient obtain a second medical opinion from a healthcare provider that is independent from the patient's current healthcare provider.

In the medical field, obtaining a second medical opinion may be necessary in certain instances. For example, a patient may want to obtain a second medical opinion when their diagnosis is complex, rare, or requires a risky medical procedure.

### SUMMARY

Embodiments of the present disclosure include a method, computer program product, and system for recommending a patient obtain a second medical opinion from a healthcare provider that is independent from the patient's current healthcare provider. A processor may collect a first set of attributes associated with a first healthcare provider, the first healthcare provider having identified a first medical opinion for treating a medical condition of a patient. The processor may identify a plurality of healthcare providers that have treated a similar medical condition for one or more other patients. The processor may compare the first set of attributes of the first healthcare provider to a plurality of sets of attributes related respectively to the plurality of healthcare providers. In response to the comparing, the processor may identify a second healthcare provider based on analyzing similarities between a second set of attributes related to the second healthcare provider and the first set of attributes related to the first healthcare provider. Once identified, the processor may provide a recommendation to the patient to obtain a second medical opinion from the second healthcare provider.

The above summary is not intended to describe each illustrated embodiment or every implementation of the present disclosure.

### BRIEF DESCRIPTION OF THE DRAWINGS

The drawings included in the present disclosure are incorporated into, and form part of, the specification. They illustrate embodiments of the present disclosure and, along with the description, serve to explain the principles of the disclosure. The drawings are only illustrative of typical embodiments and do not limit the disclosure.

FIG. 1 illustrates a block diagram of a second medical opinion recommendation system, in accordance with embodiments of the present disclosure.

FIG. 2 illustrates a flow diagram of an example process for recommending a second healthcare provider, in accordance with embodiments of the present disclosure.

FIG. 3 illustrates an example scoring model for determining a second healthcare provider, in accordance with embodiments of the present disclosure.

FIG. 4 illustrates an example graphical representation of a healthcare provider network, in accordance with embodiments of the present disclosure.

FIG. 5 illustrates a high-level block diagram of an example computer system that may be used in implementing one or more of the methods, tools, and modules, and any related functions, described herein, in accordance with embodiments of the present disclosure.

FIG. 6 depicts a cloud computing environment according to an embodiment of the present disclosure.

FIG. 7 depicts abstraction model layers according to an embodiment of the present disclosure.

While the embodiments described herein are amenable to various modifications and alternative forms, specifics thereof have been shown by way of example in the drawings and will be described in detail. It should be understood, however, that the particular embodiments described are not to be taken in a limiting sense. On the contrary, the intention is to cover all modifications, equivalents, and alternatives falling within the spirit and scope of the disclosure.

### DETAILED DESCRIPTION

Aspects of the present disclosure relate to the field of computer systems, and more particularly to a system for recommending a patient obtain a second medical opinion from a second healthcare provider that is independent from a first medical opinion given by a first healthcare provider. While the present disclosure is not necessarily limited to such applications, various aspects of the disclosure may be appreciated through a discussion of various examples using this context.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

In the medical field, obtaining a second medical opinion may be necessary in certain instances. For example, a patient may obtain a second medical when their diagnosis is complex, rare, or requires a risky medical procedure. In many instances, there may be multiple treatment options and/or differing medical opinions regarding treating the patient's given medical condition. However, the patient may not know how to utilize the proper channels necessary for obtaining an independent second medical opinion. For example, the patient may not know which other/alternative healthcare providers have dealt with similar medical conditions experienced by the patient and/or if the other healthcare providers have successfully treated those similar medical conditions. Further, the patient may not know which healthcare providers offer treatment and/or medical opinions that are different and/or independent from the first medical opinion they received from their current healthcare provider.

In many instances, the patient may want to obtain a second medical opinion from a healthcare provider that may offer an alternative treatment plan for treating their medical condition. Understanding the differences between healthcare providers and their treatment plans for certain medical conditions allows the patient to make an informed decision on obtaining a proper and/or preferred treatment plan for their given medical condition. For example, the patient's current healthcare provider may offer a less aggressive and/or standard treatment plan for the patient's current medical condition, while an alternative healthcare provider may suggest a more aggressive treatment using a novel approach that has been shown to be successful. In many instances, treatment plans between healthcare providers may differ based on various attributes, such as where the healthcare provider has received their medical education, medical training, and/or medical experience. For example, various healthcare providers may have certain differences/attributes (e.g., different educational background, academic/non-academic facility, aggressive/non-aggressive treatments, participation in different clinical trials, etc.) that correlate to the prescribed treatment plans and/or medical opinions offered for treating various medical conditions.

Embodiments of the present disclosure relate to a method, computer program product, and system that identifies and recommends an alternative healthcare provider(s) that can offer a second medical opinion(s) that is independent from a medical opinion given by a patient's current healthcare provider. In this context, healthcare provider may be any type of provider (e.g., primary care physician (PCP), doctor, surgeon, physician's assistant, etc.) that offers health services (e.g., medical treatment, a medical opinion, etc.) for treating the medical condition of the patient.

In embodiments, the system may collect a first set of attributes associated with a first healthcare provider (e.g., the patient's current healthcare provider). The first healthcare provider may be initially identified by the patient or user. For example, the patient may input the name of their current healthcare provider via a user profile on a patient device (e.g., smartphone, computer, patient portal website, etc.) that is accessible by the system. Once the first healthcare provider is identified, the system may collect the first set of attributes by analyzing one or more healthcare provider systems (e.g., healthcare network, hospital server, healthcare provider websites, claims/billing data, etc.) associated with the first healthcare provider. The first set of attributes may include any type of attribute (e.g., specialties of care, location of the provider, medical experience, medical school, geographical region of patients, types of medical conditions treated, treatment plans for various medical conditions, historic success rates of treatment plans, etc.) associated with the first healthcare provider. For example, the first set of attributes may include where a doctor went to medical school and trained during residency, what specialty of medicine the doctor practices, and treatment plans offered by the doctor for various medical conditions.

In embodiments, the system may collect and analyze the patient's electronic health records (EHRs) that are accessible through the first healthcare provider (e.g., accessed and/or stored on the first healthcare provider's network/server). The system may determine from the EHRs the patient's current medical condition and any treatment plans/medical opinions prescribed by the first healthcare provider. For example, using natural language understanding (NLU), the system may identify from the EHRs that a patient is suffering from a rare medical condition and their doctor has offered a medical opinion for treatment using a non-aggressive medicine.

Once the first healthcare provider's attributes and the patient's medical condition/treatment plan are determined, the system may identify a plurality of healthcare providers (e.g., alternative healthcare providers not associated with the patient's current healthcare provider) that have treated similar medical conditions for one or more other patients. In embodiments, the system may utilize machine learning to identify the plurality of healthcare providers from a healthcare providers network (e.g., public and/or private healthcare server(s) linked to multiple providers, public healthcare provider listings, healthcare provider's website(s), etc.). The system may access and analyze historic EHRs for the one or more other patients from each of the plurality of healthcare providers to determine which providers have treated similar medical conditions as the patient.

Once the plurality of healthcare providers that have treated similar medical conditions have been identified, the system may compare the first set of attributes of the first healthcare provider to a plurality of sets of attributes related respectively to the plurality of healthcare providers. For example, the system may use NLU to analyze the various sets of attributes associated with each of the healthcare providers to determine similarities and/or differences between the healthcare providers and the first healthcare provider. In embodiments, the system may utilize machine learning to provide a similarity score to the attributes to differentiate the healthcare providers from one another. For example, the system may use various machine learning algorithms and or distance metrics (e.g., Euclidean, Manhattan, etc.) to determine how closely related each of the plurality of healthcare providers are in comparison to the first healthcare provider (e.g., the patient's current healthcare provider).

In response to the comparing, the system will identify a second healthcare provider based on analyzing similarities between a second set of attributes related to the second healthcare provider and the first set of attributes related to the first healthcare provider. In embodiments, the system may utilize weighted preferences for scoring specific provider attributes desired by the patient. For example, a patient may want to obtain a second medical opinion from a provider that has treated similar patients relative to their geographical area and may also prefer a provider that will take a more aggressive approach to treating their medical condition. Using weighted preferences allows the system to focus on identifying healthcare providers that have these preferred attributes, while avoiding healthcare providers that do not share these preferred attributes.

In embodiments, the system may utilize weighted preferences to identify one or more attributes of the second set of attributes that are not in common with the first set of attributes to identify a second healthcare provider that is independent of the first healthcare provider. Returning to the previous example, using the weighted preferences the system will identify healthcare providers that have treated patients from the same geographical region as the current patient, but also identify providers that offer aggressive treatment plans. In this way the system will identify an alternative provider that may share similarities as their current provider (e.g., treating similar patients), but offer a different medical opinion for treatment of the medical condition (e.g. aggressive treatment vs. non-aggressive).

Once identified, the processor may provide a recommendation to the patient to obtain a second medical opinion from the identified second healthcare provider. The recommendation may be any type of communication (e.g., text message, email, phone call, notification, etc.). For example, the system may send a direct message to the patient through a patient portal recommending that the patient obtain a second medical opinion from the identified second healthcare provider. In some embodiments, the recommendation may be in the form of a ranked list that includes other suitable healthcare providers that may offer second medical opinions that are independent to the patient's current provider. The rankings of the healthcare providers may be determined according to the patient's weighted preferences and an overall similarity score of the associated attributes.

In embodiments, the patient(s) must opt into the system in order for the system to collect their information (e.g., EHRs), and the patient may determine which other users (e.g., healthcare provider(s), doctors, etc.) can utilize the collected data. For example, during an initialization process, the system may inform the patient of the types of data that it will collect (e.g., medical conditions, EHRs, treatment plans, medical opinions, etc.) and the reasons why the data is being collected. In these embodiments, the system will only start collecting the patient information upon the patient explicitly permitting the collection. Furthermore, the system may only collect the data that is necessary to provide recommendations for obtaining a second medical opinion from an identified healthcare provider. The collected data may be anonymized and/or encrypted while in use, and the data may only be maintained as needed for providing the recommendations. If the patient chooses to opt out of the system, any patient information previously collected may be permanently deleted.

The aforementioned advantages are example advantages, and not all advantages are discussed. Furthermore, embodiments of the present disclosure can exist that contain all, some, or none of the aforementioned advantages while remaining within the spirit and scope of the present disclosure.

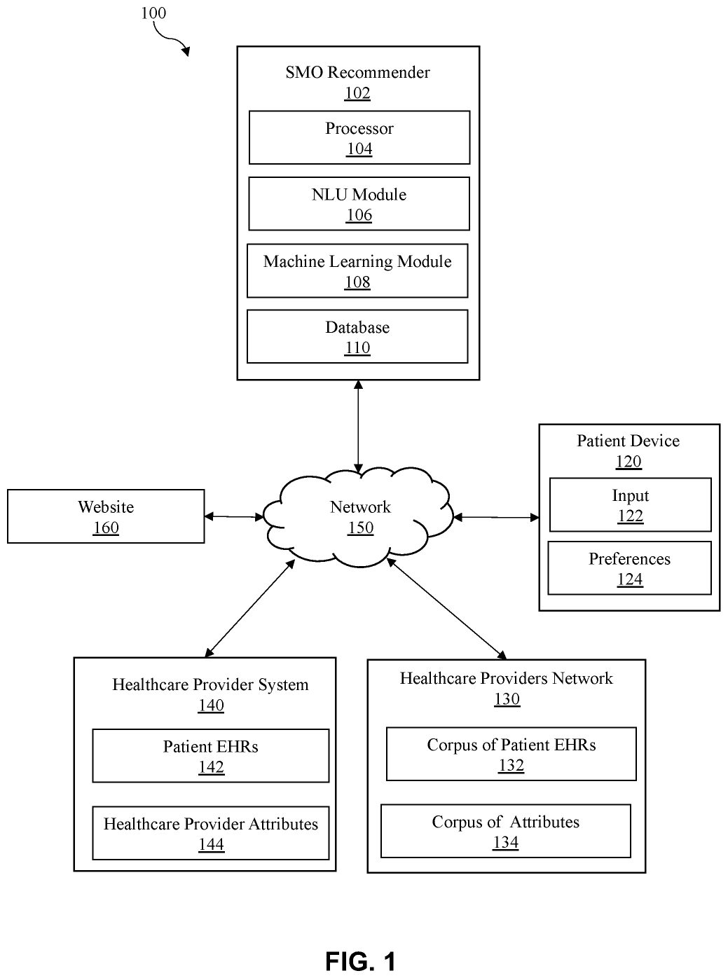

With reference now to FIG. 1, shown is a block diagram of second medical opinion (SMO) recommendation system 100, in accordance with embodiments of the present disclosure. In the illustrated embodiment, SMO recommendation system 100 includes SMO recommender 102 that is communicatively coupled to patient device 120, healthcare providers network 130, healthcare provider system 140, and website 160 via network 150.

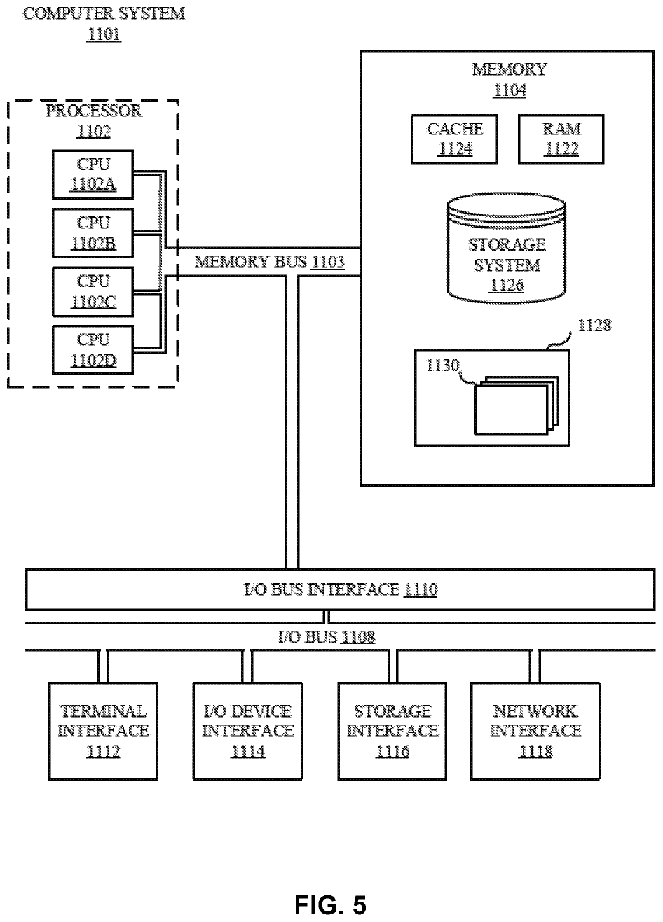

In embodiments, SMO recommender 102, patient device 120, healthcare providers network 130, healthcare provider system 140, and website 160 may be any type of computer system(s) (e.g., server, computer, mobile device, etc.) and may be substantially similar to computer system 1101 of FIG. 5. In embodiments, SMO recommender 102 may be a standalone device or located on another device, such as using cloud technology. For example, SMO recommender 102 may be a virtual application accessed by patient device 120 through a cloud computing network.

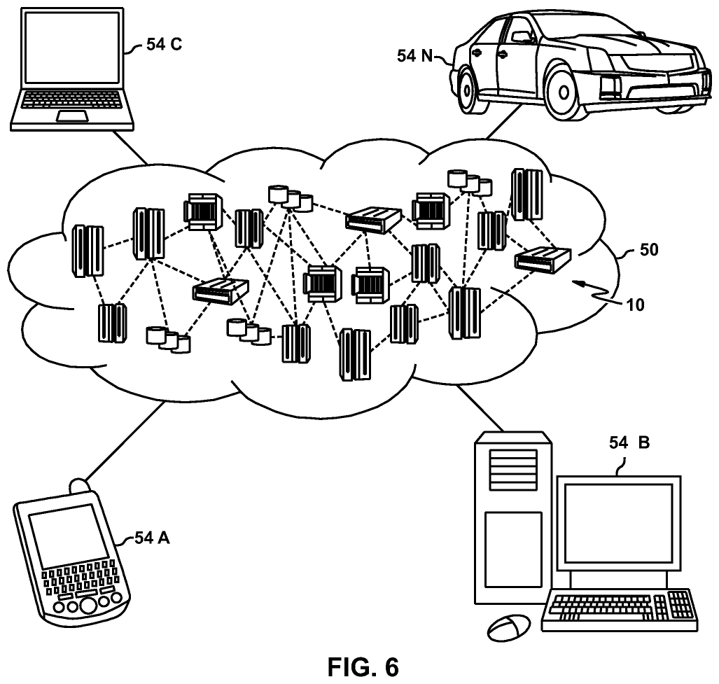

Network 150 may be any type of communication network, such as a wireless network or a cloud computing network. The network 150 may be substantially similar to, or the same as, cloud computing environment 50 described in FIG. 6. In some embodiments, the network 150 can be implemented using any number of any suitable communications media. For example, the network may be a wide area network (WAN), a local area network (LAN), a personal area network (PAN), an internet, or an intranet. In certain embodiments, the various systems may be local to each other, and communicate via any appropriate local communication medium.

For example, SMO recommender 102 may communicate with patient device 120, healthcare provider system 140, and healthcare providers network 130 using a WAN, one or more hardwire connections (e.g., an Ethernet cable) and/or wireless communication networks. In some embodiments, the various systems may be communicatively coupled using a combination of one or more networks and/or one or more local connections. For example, SMO recommender 102 may communicate with healthcare providers network 130 using a hardwired connection, while communication between patient device 120, healthcare provider system 140, and SMO recommender 102 may be through a wireless communication network.

In the illustrated embodiment, SMO recommender 102 includes processor 104, natural language understanding (NLU) module 106, machine learning module 108, and database 110.

In embodiments, SMO recommender 102 may collect, monitor, and/or analyze input 122 from patient device 120. Input 122 may include any type of data (e.g., unstructured data, text, images, etc.) that identifies the patient, the patient's current medical condition, and/or the patient's current healthcare provider (e.g., patient's primary care physician (PCP), family doctor, hospital, etc.). For example, input 122 may include the name of the patient's current healthcare provider, various patient characteristics (e.g., geographical location/region, diagnosis, medical condition, etc.), the name of the patient's insurance carrier, etc. In embodiments, SMO recommender 102 may utilize NLU module 106 to analyze input 122. For example, using NLU module 106, SMO recommender 102 may determine the patient's current healthcare provider by analyzing textual content from patient profile information (not shown) associated with patient collected from patient device 120. In embodiments, SMO recommender 102 may store and/or access input 122 related to the patient on database 110 (e.g., in the form of a user profile).

In embodiments, SMO recommender 102 may further receive preferences 124 from patient device 120. Preferences 124 may include patient preferences related to what type of attributes the patient prefers when identifying an alternative/other healthcare provider for obtaining a SMO. For example, preferences 124 may include what type of treatment plan (e.g., aggressive/non-aggressive, novel treatment approaches, etc.) the patient is comfortable with when searching for an alternative healthcare provider. In another example, preferences 124 may include location (e.g., distance from patient's location) and/or health insurance carrier preferences (e.g., in-network/out of network coverage) related to the alternative healthcare provider.

Once the patient's current healthcare provider has been identified, SMO recommender 102 may access healthcare provider system 140 associated with the current healthcare provider. Healthcare provider system 140 may be any computing system associated with a provider of health services, such as a hospital, physician's office, pharmacy, clinic, laboratory, medical imaging facility, or the like. Healthcare provider system 140 may be itself a computer that a physician (e.g., the patient's current healthcare provider, primary care physician (PCP), etc.) accesses to obtain and/or document information about the patient, or may be a server or other computing device that the physician may use to access the patient's information. For example, healthcare provider system 140 may be a tablet where a physician inputs a medical opinion for treating the patient's current medical condition.

In embodiments, healthcare provider system 140 stores and/or has access to patient electronic health records (EHRs) 142 that are specific to the patient. The patient EHRs 142 may comprise various types of patient clinical data and other patient information from a variety of different source computing systems associated with the patient's current healthcare provider, medical product providers, pharmacies, insurance companies, and the like. For example, patient EHRs data 142 may comprise a collection of clinical data for a patient obtained from hospitals, doctor offices, pharmacies, medical equipment supply companies, health insurance companies, clinics, medical imaging service providers, medical laboratories, etc. Patient EHRs 142 may comprise various treatment plans (e.g., medical opinions) offered by the patient's current healthcare provider for treating the patient's medical condition(s).

In embodiments, SMO recommender 102 collects and analyzes (e.g., using NLU module 106) patient EHRs 142 to determine the medical condition of the patient and/or a medical opinion for treatment provided to the patient. For example, the NLU module 106 may analyze patient EHRs 142 to determine the patient is suffering from a rare medical condition and has been given a suggested treatment plan by their current healthcare provider.

In embodiments, SMO recommender 102 may collect and analyze (e.g., using NLU module 106) healthcare provider attributes 144 associated with the patient's current healthcare provider accessible through healthcare provider system 140. Healthcare provider attributes 144 may include any type of attribute associated with the current healthcare provider. For example, healthcare provider attributes 144 may include general information about the current healthcare provider 140, such as where the healthcare provider received their medical education (e.g., college/university name, location, etc.), where has the healthcare provider received their medical training (e.g., residency location), which various hospitals and/or networks the healthcare provider is associated with (e.g., participation in clinical trials), types of patients the healthcare provider has treated, specialties, insurance plans accepted by the healthcare provider, etc. Healthcare provider attributes 144 may include other historical information such as historical clinical data related to various treatment plans and success rates/outcomes for patients having similar medical conditions as the patient.

In embodiments, once SMO recommender 102 has collected and analyzed the data associated with the patient (e.g., input 122, preferences 124, patient EHR 142) and the patient's current healthcare provider (e.g., healthcare provider attributes 144), the SMO recommender 102 may compare the analyzed data to a plurality of similar data sets associated with a plurality of healthcare providers (e.g., alternative healthcare providers) collected from healthcare providers network 130. In embodiments, healthcare providers network 130 may be any type of network that collects and/or stores data relative to the plurality of healthcare providers (e.g., governmental healthcare providers network, hospital network, physicians' network, healthcare provider listing, etc.).

In the illustrated embodiments, healthcare providers network 130 includes corpus of patient EHRs 132 and corpus of attributes 134. Corpus of patient EHRs 132 may comprise various types of historic patient clinical data and other historical patient information related to one or more other patients treated by the plurality of healthcare providers associated with healthcare providers network 130. In embodiments, corpus of patient EHRs 132 may comprise similar types of data as patient EHR 142. For example, the corpus of patient EHR 132 may comprise various treatment plans (e.g., medical opinions) for treating other patients that have similar medical conditions as the patient. In embodiments, the corpus of patient EHRs 132 may comprise various types of historical patient clinical data indicating successful treatment plans for curing similar medical conditions in the one or more other patients.

In embodiments, corpus of attributes 134 comprises sets of attributes related respectively to the plurality of healthcare providers. The corpus of attributes 134 may include various attributes related to each of the healthcare providers such as medical education, medical training, associated hospitals and/or networks, types of patients treated, medical conditions and related treatment plans provided to the patients, specialties, etc. The corpus of attributes 134 may be similar to healthcare provider attributes 144.

In embodiments, SMO recommender 102 collects and analyzes the corpus of patient EHR 132 using NLU module 106 to identify which of the plurality of healthcare providers from the healthcare providers network 130 have treated similar conditions to the patient. For example, the SMO recommender 102 may compare the patient's current treatment plan and medical condition from the patient EHRs 142 with a selected sample of patients from the corpus of patient EHRs 132. The identification of the similar patients may be made simply on having the same medical condition or, in order to provide a smaller sample of similar patients, SMO recommender 102 may also use demographic data associated with the patient to identify similar patients. The SMO recommender 102 may determine whether the current patient has been prescribed a similar treatment plan as other patients or if alternative treatment plans have been prescribed to patients with similar medical conditions. The SMO recommender 102 may further analyze the corpus of patient EHRs 132 to determine whether or not alternative treatment plans have been successful. If the treatment plans were not successful, the SMO recommender 102 may remove those alternative healthcare providers that prescribed unsuccessful alternative treatment plans from consideration as a chosen alternative healthcare provider for obtaining a SMO.

Once the SMO recommender 102 has determined a plurality of healthcare providers that have treated similar patients and/or medical conditions, the SMO recommender 102 may collect and analyze the corpus of attributes 134 to determine similarities and/or differences between the patient's current healthcare provider and the plurality of healthcare providers. In embodiments, the attributes of all providers may be given a similarity score by machine learning module 108. In embodiments, the machine learning module 108 may utilize preferences 124 to weight various attributes according to the patient preferences.

In some embodiments, the SMO recommender 102 may collect data from website 160 to obtain further information and/or data regarding various attributes related to each healthcare provider. Website 160 may be any internet website that contains data related to the healthcare providers. For example, website 160 may be the healthcare provider's own website where various information may be collected related to the healthcare provider. In another example, website 160 may be a social media website where unstructured data may be gathered that relates to treatment of a medical condition by a given healthcare provider. For example, the SMO recommender 102 may analyze social media comments from a former patient saying the given healthcare provider has successfully cured the former patient's medical condition. In another example, website 160 may be a medical and/or scientific journal website where the SMO recommender may access a database and collect data from various journal archives pertaining to various treatment plans, clinical trials, and the like, performed by a healthcare provider. In this way, the corpus of attributes 134 related to the healthcare providers may be supplemented based on additional information collected from website 160.

In embodiments, machine learning module 108 may utilize various machine learning techniques to compare the patient's current healthcare provider attributes 144 to the corpus of attributes 134 associated with the healthcare providers from healthcare providers network 130. For example, the machine learning module 108 may use a scoring model to assess the attributes of the plurality of healthcare providers by providing a similarity score. In embodiments, the similarity score may use weighted scores based on patient preference 124. Using the similarity score, the SMO recommender 102 may determine which healthcare provider to recommend to the patient for obtaining a SMO.

For example, patient preferences 124 may indicate that the patient wants to obtain a second medical opinion from an alternative healthcare provider that has treated similar medical conditions aggressively since their current healthcare provider is taking a non-aggressive approach. Using the similarity score, the machine learning module 108 may determine which healthcare provider would provide a SMO that is independent from the medical opinion given by the patient's current healthcare provider. In this way, SMO recommender 102 may prevent the patient from seeking a SMO from a healthcare provider that would give a similar medical opinion, allowing patients to gather alternative options for treating their medical condition.

In embodiments, machine learning module 108 may use various techniques (e.g., Jaccard index, graph distance, node structure similarity, etc.) for determining the chosen healthcare provider(s) for obtaining a SMO. For example, machine learning module 108 may measure graph distance between a minimum number of nodes (e.g., from the plurality of healthcare providers) separating the patient's current healthcare provider to identify the chosen healthcare provider.

In embodiments, Machine learning module 108 can utilize machine learning and/or deep learning, where algorithms or models can be generated by performing supervised, unsupervised, or semi-supervised training on historical data received from healthcare providers network 130. Machine learning module 108 can ingest historical treatment plans provided by the plurality of healthcare providers and success rates for treating medical conditions to generate insights, algorithms, and/or predictions related to choosing an appropriate healthcare provider to recommend to a patient for obtaining a SMO. For example, based on analyzing historic EHRs, machine learning module 108 may determine that group of healthcare providers have been more successful in treating the patient's medical condition than other healthcare providers (e.g., 70% success rate vs. 50% success rate). Using this information, the system may avoid recommending the healthcare providers that have lower success rates for treating the patient's medical condition.

In embodiments, machine learning module 108 may track which recommendations of healthcare providers have resulted in successful treatment of the current patient's medical condition after the recommendation has been given. Over time, the system may determine which healthcare providers have higher success rates when treating various medical conditions and may continually recommend those providers to patients for getting a SMO. In this way, over time, machine learning module 108 can become more accurate in properly recommending appropriate providers to improve the health of the patients utilizing the SMO recommendation system 100.

Machine learning algorithms can include, but are not limited to, decision tree learning, association rule learning, artificial neural networks, deep learning, inductive logic programming, support vector machines, clustering, Bayesian networks, reinforcement learning, representation learning, similarity/metric training, sparse dictionary learning, genetic algorithms, rule-based learning, and/or other machine learning techniques.

For example, the machine learning algorithms can utilize one or more of the following example techniques: K-nearest neighbor (KNN), learning vector quantization (LVQ), self-organizing map (SOM), logistic regression, ordinary least squares regression (OLSR), linear regression, stepwise regression, multivariate adaptive regression spline (MARS), ridge regression, least absolute shrinkage and selection operator (LASSO), elastic net, least-angle regression (LARS), probabilistic classifier, naïve Bayes classifier, binary classifier, linear classifier, hierarchical classifier, canonical correlation analysis (CCA), factor analysis, independent component analysis (ICA), linear discriminant analysis (LDA), multidimensional scaling (MDS), non-negative metric factorization (NMF), partial least squares regression (PLSR), principal component analysis (PCA), principal component regression (PCR), Sammon mapping, t-distributed stochastic neighbor embedding (t-SNE), bootstrap aggregating, ensemble averaging, gradient boosted decision tree (GBDT), gradient boosting machine (GBM), inductive bias algorithms, Q-learning, state-action-reward-state-action (SARSA), temporal difference (TD) learning, apriori algorithms, equivalence class transformation (ECLAT) algorithms, Gaussian process regression, gene expression programming, group method of data handling (GMDH), inductive logic programming, instance-based learning, logistic model trees, information fuzzy networks (IFN), hidden Markov models, Gaussian naïve Bayes, multinomial naïve Bayes, averaged one-dependence estimators (AODE), Bayesian network (BN), classification and regression tree (CART), chi-squared automatic interaction detection (CHAID), expectation-maximization algorithm, feedforward neural networks, logic learning machine, self-organizing map, single-linkage clustering, fuzzy clustering, hierarchical clustering, Boltzmann machines, convolutional neural networks, recurrent neural networks, hierarchical temporal memory (HTM), and/or other machine learning techniques.

FIG. 1 is intended to depict the representative major components of SMO recommendation system 100. In some embodiments, however, individual components may have greater or lesser complexity than as represented in FIG. 1, components other than or in addition to those shown in FIG. 1 may be present, and the number, type, and configuration of such components may vary. Likewise, one or more components shown with teaching assistant system 100 may not be present, and the arrangement of components may vary.

For example, while FIG. 1 illustrates an example SMO recommendation system 100 having a SMO recommender 102, a patient device 120, a healthcare providers network 130, healthcare provider system 140, and a website 160, suitable network architectures for implementing embodiments of this disclosure may include any number of SMO recommenders, patient devices, healthcare providers networks, healthcare providers systems, and websites. The various models, modules, systems, and components illustrated in FIG. 1 may exist, if at all, across a plurality of SMO recommenders, patient devices, healthcare provider networks, healthcare providers systems, and websites.

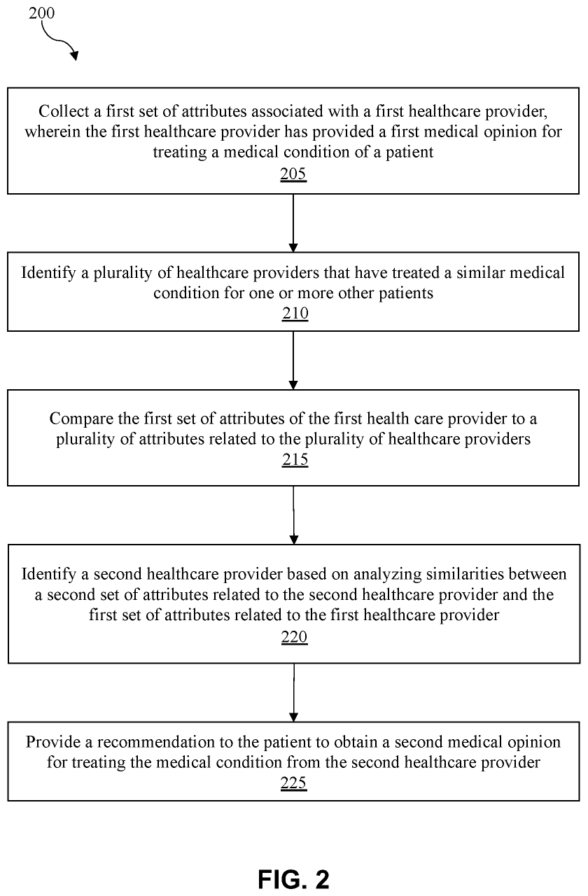

Referring now to FIG. 2, shown is a flow diagram of an example process 200 for recommending a second healthcare provider, in accordance with embodiments of the present disclosure. The process 200 may be performed by processing logic that comprises hardware (e.g., circuitry, dedicated logic, programmable logic, microcode, etc.), software (e.g., instructions run on a processor), firmware, or a combination thereof. In some embodiments, the process 200 is a computer-implemented process. The process 200 may be performed by processor 104 of SMO recommender 102 exemplified in FIG. 1.

The process 200 begins by collecting a first set of attributes associated with a first healthcare provider. This is illustrated at step 205. The first set of attributes may be collected from a healthcare provider system (e.g., healthcare provider system 140) associated with the first healthcare provider. For example, the system may collect the first set of attributes by analyzing various unstructured data obtained from the healthcare providers system such as educational background (e.g., college/university attended), medical training, specialties performed by the healthcare provider, accepted health insurance plans, patient types, and medical conditions treated by the first healthcare provider. In embodiments, the system may collect patient data (e.g., patient EHRs 142) from the first healthcare provider to determine and/or identify a medical treatment plan (e.g., medical opinion) prescribed to a patient for treating a medical condition.

The process 200 continues by identifying a plurality of healthcare providers that have treated a similar medical condition for one or more other patients. This is illustrated at step 210. In embodiments, the system may access a healthcare providers network (e.g., healthcare providers network 130) to collect and analyze data relative to treatment plans provided by the plurality of healthcare providers to the one or more other patients suffering from similar medical conditions as the current patient. For example, the system may collect and analyze the patient's EHRs 142 from healthcare provider system 140 and the corpus of patient EHRs 132 from healthcare providers network 130 to identify similar types of patients and medical conditions treated by the plurality of healthcare providers.

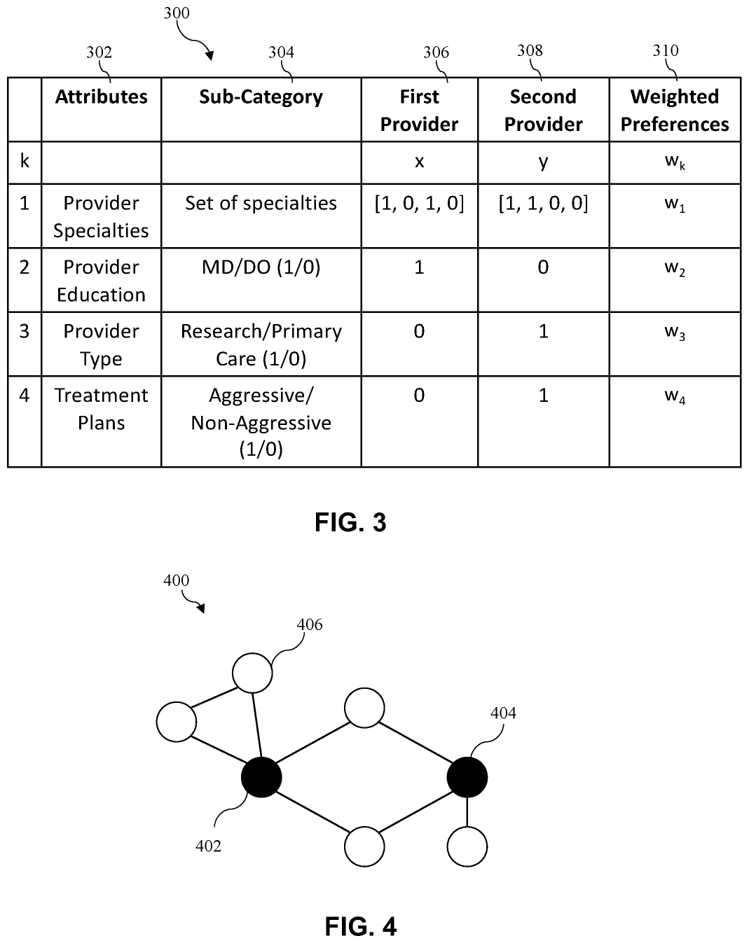

Once the plurality of healthcare providers is identified, the process 200 continues by comparing the first set of attributes related to the first healthcare provider to a plurality of sets of attributes related respectively to the plurality of healthcare providers. This is illustrated at step 215. In embodiments, the system may utilize machine learning to determine a similarity value between the first healthcare provider and the plurality of healthcare providers. For example, the system may utilize a scoring model that provides a similarity score for each attribute related to the given healthcare provider. An example of the scoring model is further described in FIG. 3.

In embodiments, the scoring model may use weighted preferences that are determined by the current patient. For example, the patient's current healthcare provider may have been medically trained in country A. However, the patient may be from a foreign country B and prefer to obtain a second medical opinion from a healthcare provider that has received their medical training in the same country B where the patient originated from. Further, the patient may prefer a healthcare provider that has treated similar patients from country B rather than country A. The system will apply a weighted preference to attributes pertaining to where the healthcare provider has trained and/or to providers that have treated patients from a similar geographic region as the current patient. In this way, the system will group healthcare providers that share these attributes and avoid providers without them. Continuing the example, the patient may also prefer that the potential healthcare providers all accept the patient's health insurance plan because the patient does not want to pay out of network costs. The system will take into account all preferences of the user when determining the potential healthcare provider.

In response to the comparing, the process 200 continues by identifying a second healthcare provider based on analyzing similarities between a second set of attributes related to the second healthcare provider and the first set of attributes related to the first healthcare provider. This is illustrated at step 220. The system may identify the second healthcare provider using various distance metrics when comparing the attributes of first healthcare provider to the second healthcare provider. Identifying the second healthcare provider using distance metrics is further described in FIG. 3 and FIG. 4.

Using the distance metrics and the weighted preferences, the system may identify a second healthcare provider that may prescribe a SMO that is independent from the first healthcare provider because the second healthcare provider has one or more attributes not in common with the first provider. Returning to the previous example, the system may identify a second healthcare provider that has trained in country B as opposed to country A, has treated patients from country B rather than country A, and that accepts the health insurance of the current patient. In this way, the second healthcare provider may offer alternative treatment plans that may be more tailored to the patient, as opposed to the patient's current provider, because the second healthcare provider has trained in the same country and treated similar patients from the same country of origin as the patient.

In some embodiments, the system may identify, from the patient's EHRs, one or more previous treatment plans that the user has undergone for the particular medical condition. For example, the patient may have already tried a treatment plan that was unsuccessful, or the patient may have previously been treated for the same medical condition. The system may consider the patient's previous history with the medical condition when suggesting a secondary healthcare provider. For example, if the patient has already unsuccessfully tried a particular treatment for the medical condition, and the system determines that a particular healthcare provider usually prescribes the particular treatment, the system may not recommend that particular healthcare provider, even if it otherwise meets the recommendation criteria. Similarly, if the patient was previously treated for the same condition successfully using a particular treatment, the system may weigh secondary healthcare providers that recommend that treatment higher, even if they have a lower score than other secondary healthcare providers (e.g., because they don't meet the geographical requirements of the patient as well as other providers).

Once the second healthcare provider is identified, the process 200 continues by providing a recommendation to the patient to obtain a second medical opinion (e.g., second medical opinion) from the second healthcare provider. This is illustrated at step 225. In embodiments, the system may output the recommendation to the patient via any type of suitable communication method. For example, the system may send a text message, email, phone call, appointment confirmation, and the like, to a patient device (e.g., patient device 120) recommending to the patient to obtain a SMO from the identified healthcare provider. In some embodiments, the recommendation may be in the form of a ranked list based on the weight preferences of the patient. For example, the list may be ranked by healthcare providers that share the most attributes preferred by the patient.

Referring now to FIG. 3, shown is an example scoring model 300 for determining a second healthcare provider, in accordance with embodiments of the present disclosure. In the illustrated embodiment, the scoring model 300 comprises a set of attributes 302 for a first healthcare provider 306 and a second healthcare provider 308. The set of attributes 302 include provider specialties, provider education, provider type, and the treatment plans offered by the first healthcare provider 306 and the second healthcare provider 308. In embodiments, each set of attributes 302 for the respective provider may be broken down into subcategories 304. For example, provider specialties are categorized into a set of specialties that are offered by the provider (e.g., general medicine, specialized medicine, oncology, specific diseases/treatments, etc.). Provider education is categorized based on type of doctor (e.g., Doctor of Medicine (MD) vs. Doctor of Osteopathic Medicine (DO)). Provider type is categorized by whether the healthcare provider is associated with a research facility or a primary care facility, and treatment plans are categorized by the type of approach taken for medical treatment (e.g., aggressive vs. non-aggressive).

In the illustrated embodiment, the system uses the scoring model 300 to score each of the respective attributes 300 (shown as k). The attributes are scored using values ranging from 0 to 1 for the first provider 306 (shown as x) and the second provider 308 (shown as y). The scoring of the attributes includes weighted preferences 310 (shown as wₖ) that may be determined by the patient. For example, a patient may have a rare medical condition that has been treated unsuccessfully at a standard primary care hospital (e.g., the first healthcare provider 306). Therefore, the patient may prefer to obtain a second medical opinion from a research hospital (e.g., the second healthcare provider 308) that is known to treat patients having the same rare medical condition aggressively. Based on the patient's preference, the system will provide a preference weight to attributes related to research facilities and aggressive treatment plans. By using the weighted preferences, the system will identify the second healthcare provider 308 using the scoring model 300. For example, the system may use distance metrics (e.g., Euclidean, Manhattan, etc.) combined with the preference weight to determine the second healthcare provider. In the illustrated embodiment, distance may be measured as:

D=Σₖ₌₁⁴wₖ|xₖ−yₖ|

Using the distance metrics, the system may identify and recommend the second healthcare provider 308 to the patient because it is both a research facility and offers aggressive treatment plans. It is noted that the illustrated embodiment is only used as an example. It is contemplated that multiple healthcare providers will be scored and compared when determining a second healthcare provider. In some embodiments, the system may use any type of machine learning/scoring model that provides a set of values (e.g., range 1-100, percentages, etc.) for comparing the attributes of the given providers.

Referring now to FIG. 4, shown is an example graphical representation of a healthcare provider network 400, in accordance with embodiments of the present disclosure. In the illustrated embodiment, the healthcare provider network 400 includes nodes signifying a first healthcare provider 402 (e.g., a patient's current healthcare provider), a plurality of alternative healthcare providers 406, and a second healthcare provider 404 (e.g., the chosen healthcare provider recommended by the system to the patient). In embodiments, the system may compare the attributes of the first healthcare provider 402 with attributes of the plurality of alternative healthcare providers 406 in order to identify the second healthcare provider 404. The comparison of attributes may be performed by machine learning to determine similarities and/or differences between the healthcare providers. The system may score the attributes (as shown in FIG. 3) of each respected healthcare provider and measure various distance metrics to determine which chosen healthcare provider to recommend to the patient for obtaining a SMO.

In embodiments, the system may utilize any type of metric (e.g., Jaccard index, minimum weighted path, node structure similarity, etc.) to determine the second healthcare provider 404. For example, once the attributes are scored, the system may determine a minimum number of nodes separating the first healthcare provider 402 and the second healthcare provider 404 from the plurality of healthcare providers 406. In another example, the system may analyze node structure similarity (e.g., centrality measurements, clustering coefficients, etc.) of the healthcare providers to determine the second healthcare provider 404. It is contemplated that other techniques may be used to determine similarities/differences between the healthcare providers and that the above examples are not meant to be limiting.

Referring now to FIG. 5, shown is a high-level block diagram of an example computer system 1101 that may be used in implementing one or more of the methods, tools, and modules, and any related functions, described herein (e.g., using one or more processor circuits or computer processors of the computer), in accordance with embodiments of the present disclosure. In some embodiments, the major components of the computer system 1101 may comprise one or more CPUs 1102, a memory subsystem 1104, a terminal interface 1112, a storage interface 1116, an I/O (Input/Output) device interface 1114, and a network interface 1118, all of which may be communicatively coupled, directly or indirectly, for inter-component communication via a memory bus 1103, an I/O bus 1108, and an I/O bus interface 1110.

The computer system 1101 may contain one or more general-purpose programmable central processing units (CPUs) 1102A, 1102B, 1102C, and 1102D, herein generically referred to as the CPU 1102. In some embodiments, the computer system 1101 may contain multiple processors typical of a relatively large system; however, in other embodiments the computer system 1101 may alternatively be a single CPU system. Each CPU 1102 may execute instructions stored in the memory subsystem 1104 and may include one or more levels of on-board cache. In some embodiments, a processor can include at least one or more of, a memory controller, and/or storage controller. In some embodiments, the CPU can execute the processes included herein (e.g., process 200).

System memory subsystem 1104 may include computer system readable media in the form of volatile memory, such as random access memory (RAM) 1122 or cache memory 1124. Computer system 1101 may further include other removable/non-removable, volatile/non-volatile computer system data storage media. By way of example only, storage system 1126 can be provided for reading from and writing to a non-removable, non-volatile magnetic media, such as a “hard drive.” Although not shown, a magnetic disk drive for reading from and writing to a removable, non-volatile magnetic disk (e.g., a “floppy disk”), or an optical disk drive for reading from or writing to a removable, non-volatile optical disc such as a CD-ROM, DVD-ROM or other optical media can be provided. In addition, memory subsystem 1104 can include flash memory, e.g., a flash memory stick drive or a flash drive. Memory devices can be connected to memory bus 1103 by one or more data media interfaces. The memory subsystem 1104 may include at least one program product having a set (e.g., at least one) of program modules that are configured to carry out the functions of various embodiments.

Although the memory bus 1103 is shown in FIG. 5 as a single bus structure providing a direct communication path among the CPUs 1102, the memory subsystem 1104, and the I/O bus interface 1110, the memory bus 1103 may, in some embodiments, include multiple different buses or communication paths, which may be arranged in any of various forms, such as point-to-point links in hierarchical, star or web configurations, multiple hierarchical buses, parallel and redundant paths, or any other appropriate type of configuration. Furthermore, while the I/O bus interface 1110 and the I/O bus 1108 are shown as single units, the computer system 1101 may, in some embodiments, contain multiple I/O bus interfaces 1110, multiple I/O buses 1108, or both. Further, while multiple I/O interface units are shown, which separate the I/O bus 1108 from various communications paths running to the various I/O devices, in other embodiments some or all of the I/O devices may be connected directly to one or more system I/O buses.

In some embodiments, the computer system 1101 may be a multi-user mainframe computer system, a single-user system, or a server computer or similar device that has little or no direct user interface, but receives requests from other computer systems (clients). Further, in some embodiments, the computer system 1101 may be implemented as a desktop computer, portable computer, laptop or notebook computer, tablet computer, pocket computer, telephone, smart phone, network switches or routers, or any other appropriate type of electronic device.

It is noted that FIG. 5 is intended to depict the representative major components of an exemplary computer system 1101. In some embodiments, however, individual components may have greater or lesser complexity than as represented in FIG. 5, components other than or in addition to those shown in FIG. 5 may be present, and the number, type, and configuration of such components may vary.

One or more programs/utilities 1128, each having at least one set of program modules 1130 may be stored in memory subsystem 1104. The programs/utilities 1128 may include a hypervisor (also referred to as a virtual machine monitor), one or more operating systems, one or more application programs, other program modules, and program data. Each of the operating systems, one or more application programs, other program modules, and program data or some combination thereof, may include an implementation of a networking environment. Programs/utilities 1128 and/or program modules 1130 generally perform the functions or methodologies of various embodiments.

It is understood in advance that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

Referring now to FIG. 6, illustrative cloud computing environment 50 is depicted. As shown, cloud computing environment 50 comprises one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 6 are intended to be illustrative only and that computing nodes 10 and cloud computing environment 50 can communicate with any type of computerized device over any type of network and/or network addressable connection (e.g., using a web browser).

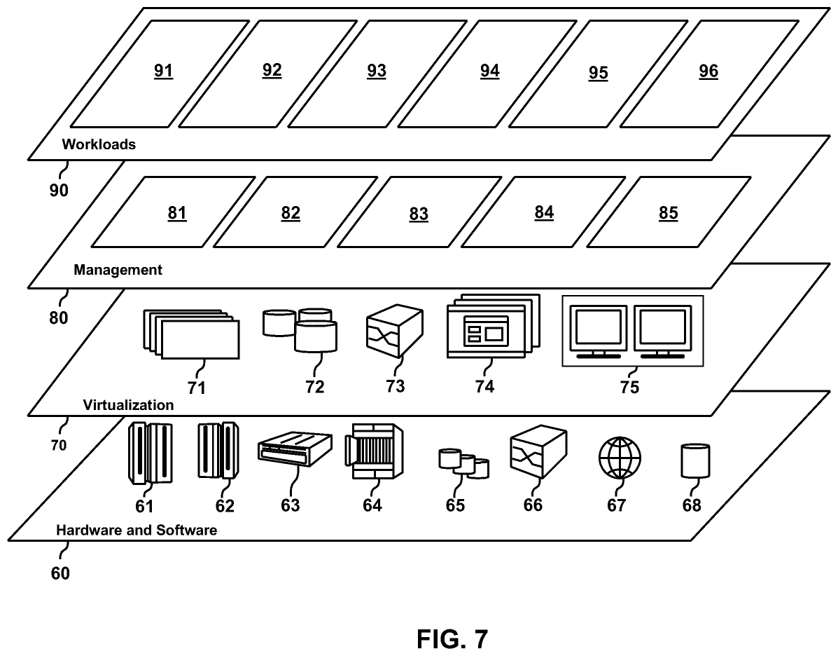

Referring now to FIG. 7, a set of functional abstraction layers provided by cloud computing environment 50 (FIG. 6) is shown. It should be understood in advance that the components, layers, and functions shown in FIG. 7 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and SMO recommender software 68 in relation to the SMO recommender 102 of FIG. 1.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and mobile desktops 96.

As discussed in more detail herein, it is contemplated that some or all of the operations of some of the embodiments of methods described herein may be performed in alternative orders or may not be performed at all; furthermore, multiple operations may occur at the same time or as an internal part of a larger process.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

What is claimed is:

1. A computer-implemented method comprising:
collecting a first set of attributes associated with a first healthcare provider, wherein the first healthcare provider has provided a first medical opinion for treating a medical condition of a patient;
identifying a plurality of healthcare providers that have treated a similar medical condition for one or more other patients;
comparing the first set of attributes of the first healthcare provider to a plurality of sets of attributes related respectively to the plurality of healthcare providers;
in response to the comparing, identifying a second healthcare provider based on analyzing similarities between a second set of attributes related to the second healthcare provider and the first set of attributes related to the first healthcare provider; and
providing a recommendation to the patient to obtain a second medical opinion for treating the medical condition from the second healthcare provider.

2. The computer-implemented method of claim 1, wherein the first set of attributes and the second set of attributes include one or more attributes selected from the group consisting of:
geographical region of patients;
educational background of a given provider;
a plurality of medical opinions prescribed for treating a plurality of medical conditions;
accepted insurance plans; and
historic success rates for treatments related to various medical conditions.

3. The computer-implemented method of claim 1, wherein the second set of attributes includes at least one attribute not in common with the first set of attributes.

4. The computer-implemented method of claim 3, wherein the at least one attribute not in common with the first set of attributes is an educational background of the second healthcare provider.

5. The computer-implemented method of claim 1, wherein comparing the first set of attributes of the first healthcare provider to the plurality of sets of attributes related to the plurality of healthcare providers comprises calculating, using machine learning, a set of weights for the first set of attributes and the plurality of sets of attributes based in part on patient preference.

6. The computer-implemented method of claim 1, wherein the recommendation comprises a ranked list of healthcare providers based on patient preference, wherein the second healthcare provider is ranked as the most preferred.

7. The computer-implemented method of claim 1, wherein identifying the plurality of healthcare providers that have treated the similar medical condition for one or more other patients comprises analyzing, using natural language understanding, electronic health records of the patient and the one or more other patients.

8. The computer-implement method of claim 1, wherein the recommendation is sent to a patient device associated with the patient.

9. A system comprising:
a processor; and
a computer-readable storage medium communicatively coupled to the processor and storing program instructions which, when executed by the processor, cause the processor to perform a method comprising:
collecting a first set of attributes associated with a first healthcare provider, wherein the first healthcare provider has provided a first medical opinion for treating a medical condition of a patient;
identifying a plurality of healthcare providers that have treated a similar medical condition for one or more other patients;
comparing the first set of attributes of the first healthcare provider to a plurality of sets of attributes related respectively to the plurality of healthcare providers;
in response to the comparing, identifying a second healthcare provider based on analyzing similarities between a second set of attributes related to the second healthcare provider and the first set of attributes related to the first healthcare provider; and
providing a recommendation to the patient to obtain a second medical opinion for treating the medical condition from the second healthcare provider.

10. The system of claim 9, wherein the first set of attributes and the second set of attributes include one or more attributes selected from the group consisting of:
geographical region of patients;
educational background of a given healthcare provider;
a plurality of medical opinions prescribed for treating a plurality of medical conditions;
accepted insurance plans; and
historic success rates for treatments related to various medical conditions.

11. The system of claim 9, wherein the second set of attributes includes at least one attribute not in common with the first set of attributes.

12. The system of claim 11, wherein the at least one attribute not in common with the first set of attributes is a geographical region of patients treated by the second healthcare provider.

13. The system of claim 9, wherein comparing the first set of attributes of the first healthcare provider to the plurality of sets of attributes related to the plurality of healthcare providers comprises calculating, using machine learning, a set of weights for the first set of attributes and the plurality of sets of attributes based in part on patient preference.

14. The system of claim 9, wherein identifying the plurality of healthcare providers that have treated the similar medical condition for one or more other patients comprises analyzing, using natural language understanding, electronic health records of the patient and the one or more other patients.

15. A computer program product comprising a computer-readable storage medium having program instructions embodied therewith, the program instructions executable by a processor to cause the processor to perform a method comprising:
collecting a first set of attributes associated with a first healthcare provider, wherein the first healthcare provider has provided a first medical opinion for treating a medical condition of a patient;
identifying a plurality of healthcare providers that have treated a similar medical condition for one or more other patients;
comparing the first set of attributes of the first healthcare provider to a plurality of sets of attributes related respectively to the plurality of healthcare providers;
in response to the comparing, identifying a second healthcare provider based on analyzing similarities between a second set of attributes related to the second healthcare provider and the first set of attributes related to the first healthcare provider; and
providing a recommendation to the patient to obtain a second medical opinion for treating the medical condition from the second healthcare provider.

16. The computer program product of claim 15, wherein the first set of attributes and the second set of attributes include one or more attributes selected from the group consisting of:
geographical region of patients;
educational background of a given healthcare provider;
a plurality of medical opinions prescribed for treating a plurality of medical conditions;
accepted insurance plans; and
historic success rates for treatments related to various medical conditions.

17. The computer program product of claim 15, wherein the second set of attributes includes at least one attribute not in common with the first set of attributes.

18. The computer program product of claim 17, wherein the at least one attribute not in common with the first set of attributes is a plurality of medical opinions prescribed for treating the similar medical condition by the second healthcare provider.

19. The computer program product of claim 15, wherein comparing the first set of attributes of the first healthcare provider to the plurality of sets of attributes related to the plurality of healthcare providers comprises calculating, using machine learning, a set of weights for the first set of attributes and the plurality of sets of attributes based in part on patient preference.

20. The computer program product of claim 15, wherein identifying the plurality of healthcare providers that have treated the similar medical condition for one or more other patients comprises analyzing, using natural language understanding, electronic health records of the patient and the one or more other patients.
